(19) United States
(12) Patent Application Publication
(10) Pub. No.: US 2023/0248705 A1
(43) Pub. Date: Aug. 10, 2023

(54) ADMINISTRATION OF MODULATORS OF 5-HT AND / OR AMPA RECEPTORS FOR TREATING NEUROLOGICAL CONDITIONS

(71) Applicant: THE ROYAL INSTITUTION FOR THE ADVANCEMENT OF LEARNING/MCGILL UNIVERSITY, Montreal (CA)

(72) Inventors: Gabriella GOBBI, Montreal (CA); Danilo DE GREGORIO, Montreal (CA)

(21) Appl. No.: 18/102,296

(22) Filed: Jan. 27, 2023

Related U.S. Application Data
(63) Continuation of application No. PCT/IB2021/000494, filed on Jul. 28, 2021.
(60) Provisional application No. 63/058,374, filed on Jul. 29, 2020.

(51) Int. Cl. A61K 31/437; A61P 25/22

(52) U.S. Cl. CPC A61K 31/437; A61P 25/22

(57) ABSTRACT

Disclosed herein are methods of treating neuropsychiatric and cognitive diseases, disorders, or conditions, such as social disfunction, with repeated doses of a modulator of the AMPA receptor and/or the 5-HT_{2A} receptor.

### CROSS-REFERENCE

This application is a bypass continuation of International Application No PCT/IB2021/000494, filed Jul. 28, 2021, which claims the benefit of U.S. Provisional Application No. 63/058,374, filed Jul. 29, 2020, each of which is incorporated herein by reference in its entirety.

### BACKGROUND

The α-amino-3-hydroxy-5-methyl-4-isoxazolepropionic acid (AMPA) receptor is a subtype of the ionotropic glutamate receptor coupled to ion channels that modulate cell excitability by gating the flow of calcium and sodium ions into the cell. The serotonin (5-HT) receptors are a group of G protein-coupled receptors (GPCRs) and ligand-gated ion channels found in the central and peripheral nervous systems. Activation of AMPA receptors and/or 5-HT receptors can substantially influence brain function, such as, to treat psychiatric diseases.

### SUMMARY

Provided herein in some embodiments is a method for treating or managing a mental, a behavioral, or a neuropsychiatric condition, or, the symptoms thereof, in an individual in need thereof, comprising administering to the individual a (e.g., therapeutically) effective amount of one or more modulator of an α-amino-3-hydroxy-5-methyl-5-isoxazolepropionic acid (AMPA) receptor and/or a 5-hydroxytryptamine (e.g., 5-hydroxytryptamine 2A (5-HT_{2A})) receptor (e.g., lysergic acid (LSD)), or a pharmaceutically acceptable salt, solvate, metabolite, derivative, or prodrug thereof (e.g., for an extended period of time (e.g., for repeating days)).

In some embodiments, the method comprises administering to the individual a (e.g., therapeutically) effective amount of one or more modulator of an AMPA receptor and a 5-HT (e.g., a 5-HT_{2A}) receptor (e.g., lysergic acid (LSD)), or a pharmaceutically acceptable salt, solvate, metabolite, derivative, or prodrug thereof (e.g., for an extended period of time (e.g., for repeating days)).

In certain embodiments, (e.g., pharmaceutical) compositions provided herein and/or methods provided herein are configured to provide a prolonged and/or repeated exposure in an individual to a modulator of the AMPA receptor and/or the 5-HT_{2A} receptor (e.g., lysergic acid (LSD)), or a pharmaceutically acceptable salt, solvate, metabolite, derivative, or prodrug thereof.

In some embodiments, a modulator of the AMPA receptor and/or the 5-HT_{2A} receptor (e.g., lysergic acid (LSD)), or a pharmaceutically acceptable salt, solvate, metabolite, derivative, or prodrug thereof is provided to an individual at a first time point and a second time point. In certain embodiments, a composition provided herein comprises a first dosage form comprising a modulator of the AMPA receptor and/or the 5-HT_{2A} receptor (e.g., lysergic acid (LSD)), or a pharmaceutically acceptable salt, solvate, metabolite, derivative, or prodrug thereof, and a second dosage form comprising a modulator of the AMPA receptor and/or the 5-HT_{2A} receptor (e.g., lysergic acid (LSD)), or a pharmaceutically acceptable salt, solvate, metabolite, derivative, or prodrug thereof (e.g., wherein the modulator of the AMPA receptor and/or the 5-HT_{2A} receptor (e.g., lysergic acid (LSD)) of the first and second dosage forms are the same or different). In some embodiments, a first dosage form is administered on a first day and a second dosage form is administered on a second day that is at least 1 day after the first day (e.g., at least 2 days, at least 3 days, at least 4 days, at least 5 days, at least 6 days, or at least 7 days after the first day). In some embodiments, additional dosage forms are also administered, such as on intervening days and/or subsequent to administration of the second dosage form.

In certain embodiments, a composition (or dosage form) provided herein is an immediate or a controlled (e.g., an extended) release composition (or dosage form)). In some instances, the immediate or controlled (e.g., an extended) release composition (or dosage form), or component thereof, provides exposure to a modulator of the AMPA receptor and/or the 5-HT_{2A} receptor for an (e.g., extended) period of time, such as for at least 30 minutes, at least 1 hour, at least 2 hours, or longer. In some instances, the exposure is at or above a minimum effective (e.g., serum) concentration of an active modulator of the AMPA receptor and/or the 5-HT_{2A} receptor).

In some instances, repeated and/or prolonged delivery of a modulator of the AMPA receptor and/or the 5-HT_{2A} receptor, such as via a composition provided herein, provides certain behavioral results (e.g., behavioral effects), such as distinct from certain results seen when the modulator of the AMPA receptor and/or the 5-HT_{2A} receptor is administered as a single dose. For example, in some instances, repeated administration of a modulator of the AMPA receptor and/or the 5-HT_{2A} receptor provides for prolonged effects, such as prolonged improvement in cognitive capability, neurodegenerative effects, and/or social behavior (SB). In certain instances, repeated and prolonged delivery of modulator of the AMPA receptor and/or the 5-HT_{2A} receptor results in an improved ability to respond to stressors. In some instances, repeated and/or prolonged delivery of a modulator of the AMPA receptor and/or the 5-HT_{2A} receptor, such as via a composition provided herein, results in a decrease of adverse events, such as hallucinations (e.g. tolerance), in an individual receiving the composition or dosage form.

In some embodiments, provided herein is a method comprising maintaining a level of an active modulator of the AMPA receptor and/or the 5-HT_{2A} receptor at or above a minimum therapeutically effective threshold for more than or equal to two hours.

In some instances, the minimum therapeutically effective threshold is below an unwanted (hallucinogenic) threshold (e.g., below a sub-psychotic hallucinogenic threshold), such as a level whereby an unwanted (hallucinogenic) effect (e.g., a psychotic-like hallucinogenic effect) results from at least a first dose. In some embodiments, the minimum therapeutically effective threshold is above a hallucinogenic threshold, such as a level whereby the hallucinogenic effect results from at least a first dose, wherein the hallucinogenic effect reduces over repeat dosing of the active 5-HT receptor agonist in the individual. In some embodiments, the minimum therapeutically effective threshold occurs within a window of operability, wherein therapeutic and hallucinogenic outcomes overlap for at least one dose, and prolonged dosing results in a transition from adverse events, such as hallucinations, to reduced or negligible adverse events while maintaining a therapeutic outcome.

In some embodiments, provided herein is a method for treating a mental, behavioral, or neuropsychiatric condition, or the symptoms thereof in an individual in need thereof. In some embodiments, the method is for managing a mental, a behavioral, or a neuropsychiatric condition, or the symptoms thereof in an individual in need thereof. In some embodiments, the method is for treating and managing a mental, behavioral, or neuropsychiatric condition, or the symptoms thereof in an individual in need thereof. In some embodiments, the individual is suffering from or susceptible to the mental, a behavioral, or a neuropsychiatric condition. In certain instances, the symptoms of the mental, a behavioral, or a neuropsychiatric condition are physical, behavioral, emotional, mental, or a combination thereof.

In some embodiments, the mental, the behavioral, or the neuropsychiatric condition is any disease or disorder provided herein. In some embodiments, the mental, the behavioral, or the neuropsychiatric condition is a chronic condition. In some embodiments, the mental, the behavioral, or the neuropsychiatric condition is a Diagnostic and Statistical Manual of Mental Disorders (DSM-5) category disease or disorder. In some embodiments, the mental, the behavioral, or the neuropsychiatric condition is a non-DSM-5 category disease or disorder. In some embodiments, the mental, the behavioral, or the neuropsychiatric condition is selected from the group consisting of anxiety (e.g., social anxiety, anxiety disorders, generalized anxiety, substance-induced anxiety, or stress-induced anxiety), fear, phobia (e.g., a social phobia), constructive impulsivity, autism spectrum disorder (ASD), or depression, or the like. In some embodiments, the mental, the behavioral, or the neuropsychiatric condition is a social behavior condition (e.g., autism spectrum disorder (ASD)).

In some embodiments, provided herein is a method comprising increasing social interaction or decreasing social dysfunction in an individual in need thereof. In some embodiments, the method comprises increasing social interaction or decreasing social dysfunction in an individual suffering or susceptible to anxiety (e.g., social anxiety, anxiety disorders, generalized anxiety), fear, phobia (e.g., a social phobia), constructive impulsivity, autism spectrum disorder (ASD), or depression).

In some embodiments, provided herein is a method comprising treating or managing a neurodegenerative condition (e.g., Alzheimer's disease) or a neurodevelopmental condition (e.g., autism spectrum disorder (ASD)), or the symptoms thereof, in an individual in need thereof. In some embodiments, provided herein is a method comprising treating or managing a neurodegenerative condition or a neurodevelopmental condition, or the symptoms thereof, that affects cognition in an individual suffering from or susceptible to the neurodegenerative condition or the neurodevelopmental condition. In some embodiments, the condition is Rett Syndrome or Fragile X Syndrome.

In some embodiments, the method comprises administering the active modulator of the AMPA receptor and/or the 5-HT_{2A} receptor in the individual for a period of time sufficient to treat or manage the mental, the behavioral, or the neuropsychiatric condition(s), or the symptoms thereof, of the individual. In some embodiments, the method comprises administering the active modulator of the AMPA receptor and/or the 5-HT_{2A} receptor in the individual for a period of time sufficient to increase social interaction or decrease social dysfunction of the individual. In some embodiments, the method comprises administering the active modulator of the AMPA receptor and/or the 5-HT_{2A} receptor in the individual for a period of time sufficient to treat or manage the neurodegenerative condition or the neurodevelopmental condition of the individual.

In some embodiments, the mental, the behavioral, or the neuropsychiatric condition is a social behavior (e.g., a social anxiety disorder).

In some embodiments, the mental, the behavioral, or the neuropsychiatric condition is an anxiety disorder or a depression disorder.

In some embodiments, the mental, the behavioral, or the neuropsychiatric condition is depression.

In some embodiments, the mental, the behavioral, or the neuropsychiatric condition is anxiety.

In some embodiments, the mental, the behavioral, or the neuropsychiatric condition is social anxiety.

In some embodiments, the method comprises administering the active modulator of the AMPA receptor and/or the 5-HT_{2A} receptor in the individual for a period of more than or equal to more than or equal to 1 day, more than or equal to 2 days, more than or equal to 3 days, more than or equal to 4 days, more than or equal to 5 days, more than or equal to 6 days, or more than or equal to 7 days. In some embodiments, the method comprises administering the 5-HT receptor agonist in the individual for a period of more than 1 day.

Provided in certain instances herein is a method for treating or managing a mental, a behavioral, or a neuropsychiatric condition, or the symptoms thereof, in an individual in need thereof (e.g., in an individual suffering from or susceptible to the mental, the behavioral, or the neuropsychiatric condition), comprising administering to the individual an effective amount of a modulator of the AMPA receptor and/or the 5-HT_{2A} receptor (e.g., lysergic acid (LSD)), or a pharmaceutically acceptable salt, solvate, metabolite, derivative, or prodrug thereof, on a first day. In some embodiments, the method comprises administering to the individual the effective amount of the modulator of the AMPA receptor and/or the 5-HT_{2A} receptor (e.g., LSD), or a pharmaceutically acceptable salt, solvate, metabolite, derivative, or prodrug thereof, on a second day. In some embodiments, the second day is at least one day after (e.g., at least two days after, at least three days after, at least four days after, or at least 7 days after) the first day.

In some instances, the method comprises administering at least one dose of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof).

In some instances, the method comprises acute administration of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof).

In some instances, the method comprises sub-chronic administration of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof).

In some instances, the method comprises chronic administration of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof).

Provided in certain instances herein is a method for increasing social interaction or decreasing social dysfunction in an individual (e.g., in an individual suffering from or susceptible to anxiety (e.g., social anxiety, anxiety disorders, generalized anxiety), fear, phobia (e.g., a social phobia), constructive impulsivity, autism spectrum disorder (ASD), or depression), comprising administering to the individual an effective amount of a modulator of the AMPA receptor and/or the 5-HT_{2A} receptor (e.g., lysergic acid (LSD)), or a pharmaceutically acceptable salt, solvate, metabolite, derivative, or prodrug thereof, on a first day. In some embodiments, the method comprises administering to the individual the effective amount of the modulator of the AMPA receptor and/or the 5-HT_{2A} receptor (e.g., LSD), or a pharmaceutically acceptable salt, solvate, metabolite, derivative, or prodrug thereof, on a second day. In some embodiments, the second day is at least one day after (e.g., at least two days after, at least three days after, at least four days after, or at least 7 days after) the first day.

Provided in certain instances herein is a method for treating and/or managing a neurodegenerative condition or a neurodevelopmental condition (e.g., that affects cognition), or the symptoms thereof, in an individual in need thereof (e.g., in an individual suffering from or susceptible to the neurodegenerative condition or the neurodevelopmental condition), comprising administering to the individual an effective amount of a modulator of the AMPA receptor and/or the 5-HT_{2A} receptor (e.g., lysergic acid (LSD)), or a pharmaceutically acceptable salt, solvate, metabolite, derivative, or prodrug thereof, on a first day. In some embodiments, the method comprises administering to the individual the effective amount of the modulator of the AMPA receptor and/or the 5-HT_{2A} receptor (e.g., LSD), or a pharmaceutically acceptable salt, solvate, metabolite, derivative, or prodrug thereof, on a second day. In some embodiments, the second day is at least one day after (e.g., at least two days after, at least three days after, at least four days after, or at least 7 days after) the first day.

In some embodiments, the method further comprises administering to the individual an effective (e.g., a therapeutically effective) amount of the modulator of the AMPA receptor and/or the 5-HT_{2A} receptor (e.g., LSD), or a pharmaceutically acceptable salt, solvate, metabolite, derivative, or prodrug thereof on a third day. In some embodiments, the third day is a day between the first and second day. In some embodiments, the third day is a day after the second day.

In some embodiments, the method further comprises administering to the individual an effective (e.g., a therapeutically effective) amount of the modulator of the AMPA receptor and/or the 5-HT_{2A} receptor (e.g., LSD), or a pharmaceutically acceptable salt, solvate, metabolite, derivative, or prodrug thereof on a fourth day. In some embodiments, the fourth day is a day between the first and third day. In some embodiments, the fourth day is a day between the first and second day. In some embodiments, the fourth day is a day after the first day. In some embodiments, the fourth day is a day after the second day. In some embodiments, the fourth day is a day after the third day.

In some embodiments, the method further comprises administering to the individual an effective (e.g., a therapeutically effective) amount of the modulator of the AMPA receptor and/or the 5-HT_{2A} receptor (e.g., LSD), or a pharmaceutically acceptable salt, solvate, metabolite, derivative, or prodrug thereof on a fifth day. In some embodiments, the fifth day is a day between the first and fourth day. In some embodiments, the fifth day is a day between the first and third day. In some embodiments, the fifth day is a day between the first and second day. In some embodiments, the fifth day is a day after the first day. In some embodiments, the fifth day is a day after the second day. In some embodiments, the fifth day is a day after the third day. In some embodiments, the fifth day is a day after the fourth day.

In some embodiments, the method further comprises administering to the individual an effective (e.g., a therapeutically effective) amount of the modulator of the AMPA receptor and/or the 5-HT_{2A} receptor (e.g., LSD), or a pharmaceutically acceptable salt, solvate, metabolite, derivative, or prodrug thereof on a sixth day. In some embodiments, the sixth day is a day between the first and fifth day. In some embodiments, the sixth day is a day between the first and fourth day. In some embodiments, the sixth day is a day between the first and third day. In some embodiments, the sixth day is a day between the first and second day. In some embodiments, the sixth day is a day after the first day. In some embodiments, the sixth day is a day after the second day. In some embodiments, the sixth day is a day after the third day. In some embodiments, the sixth day is a day after the fourth day. In some embodiments, the sixth day is a day after the fifth day.

In some embodiments, the method further comprises administering to the individual an effective (e.g., a therapeutically effective) amount of the modulator of the AMPA receptor and/or the 5-HT_{2A} receptor (e.g., LSD), or a pharmaceutically acceptable salt, solvate, metabolite, derivative, or prodrug thereof on a seventh day. In some embodiments, the seventh day is a day between the first and sixth day. In some embodiments, the seventh day is a day between the first and fifth day. In some embodiments, the seventh day is a day between the first and fourth day. In some embodiments, the seventh day is a day between the first and third day. In some embodiments, the seventh day is a day between the first and second day. In some embodiments, the seventh day is a day after the first day. In some embodiments, the seventh day is a day after the second day. In some embodiments, the seventh day is a day after the third day. In some embodiments, the seventh day is a day after the fourth day. In some embodiments, the seventh day is a day after the fifth day. In some embodiments, the seventh day is a day after the sixth day.

In some embodiments, the method further comprises administering to the individual an effective (e.g., a therapeutically effective) amount of the modulator of the AMPA receptor and/or the 5-HT_{2A} receptor (e.g., LSD), or a pharmaceutically acceptable salt, solvate, metabolite, derivative, or prodrug thereof on a first day, a second day, a third day, a fourth day, a fifth day, a sixth day, and a seventh day. In some embodiments, each of the first day, the second day, the third day, the fourth day, the fifth day, the sixth day, and the seventh day are consecutive or repeating days. In some embodiments, the first day is no less than one day apart from the second day, the second day is no less than one day apart from the third day, the third day is no less than one day apart from the fourth day, the fourth day is no less than one day apart from the fifth day, the fifth day is no less than one day apart from the sixth day, and the sixth day is no less than one day apart from the seventh day.

In some embodiments, the method comprises administering a repeated dose (e.g., over an extended period of time (e.g., daily for a week, every other day for a week, two times a week, once a week, bi-weekly, or the like)) of the modulator of the AMPA receptor and/or the 5-HT_{2A} receptor, or the pharmaceutically acceptable salt, solvate, metabolite, derivative, or prodrug thereof, to the individual in need thereof.

In some embodiments, the effective amount (e.g., the therapeutically effective amount) of the modulator of the AMPA receptor and/or the 5-HT_{2A} receptor is administered at least once daily for at least two days. In some embodiments, the effective amount (e.g., the therapeutically effective amount) of the modulator of the AMPA receptor and/or the 5-HT_{2A} receptor is administered at least daily for a week or more. In some embodiments, the effective amount (e.g., the therapeutically effective amount) of the modulator of the AMPA receptor and/or the 5-HT_{2A} receptor is administered at least daily for a month or more. In some embodiments, the effective amount (e.g., the therapeutically effective amount) of the modulator of the AMPA receptor and/or the 5-HT_{2A} receptor is administered at least every other day for a week or more. In some embodiments, the effective amount (e.g., the therapeutically effective amount) of the modulator of the AMPA receptor and/or the 5-HT_{2A} receptor is administered at least every other day for a month or more. In some embodiments, the effective amount (e.g., the therapeutically effective amount) of the modulator of the AMPA receptor and/or the 5-HT_{2A} receptor is administered at least two times a week for a month or more. In some embodiments, the effective amount (e.g., the therapeutically effective amount) of the modulator of the AMPA receptor and/or the 5-HT_{2A} receptor is administered at least once a week for a month or more. In some embodiments, the effective amount (e.g., the therapeutically effective amount) of the modulator of the AMPA receptor and/or the 5-HT_{2A} receptor is administered at least bi-weekly for a month or more. In some embodiments, the effective amount (e.g., the therapeutically effective amount) of the modulator of the AMPA receptor and/or the 5-HT_{2A} receptor (e.g. LSD) is administered at least once a day, once a week or bi-weekly for at least 12 weeks or more.

In some embodiments, the modulator of the AMPA receptor and/or the 5-HT_{2A} receptor is a hallucinogenic compound, or a pharmaceutically acceptable salt, solvate, metabolite, derivative, or prodrug thereof. In some embodiments, the modulator of the AMPA receptor and/or the 5-HT_{2A} receptor is an ergoline, or a pharmaceutically acceptable salt, solvate, metabolite, derivative, or prodrug thereof. In some embodiments, the modulator of the AMPA receptor and/or the 5-HT_{2A} receptor is LSD, or a pharmaceutically acceptable salt, solvate, metabolite, derivative, or prodrug thereof.

In some embodiments, the hallucinogenic compound produces a hallucinogenic effect in the individual. In some embodiments, the hallucinogenic compound, or a pharmaceutically acceptable salt, solvate, metabolite, derivative, or prodrug thereof, produces an adverse event or a clinically important effect in the individual in need thereof at or above the hallucinogenic threshold of the hallucinogenic compound. In some embodiments, the clinically important effect is a clinically important impairment of the individual, altered perception, altered cognition, impaired attention, drowsiness, and/or confusion. In some embodiments, the altered perception in the individual is a visual perception alteration, an auditory perception alteration, bodily perception alteration, a temporal perception alteration, or a spatial perception alteration.

In some instances, the modulator of the AMPA receptor and/or the 5-HT_{2A} receptor, or the pharmaceutically acceptable salt, solvate, metabolite, derivative, or prodrug thereof, is administered to the individual at an amount of at most 200 micrograms (mcg). In some instances, the modulator of the AMPA receptor and/or the 5-HT_{2A} receptor, or the pharmaceutically acceptable salt, solvate, metabolite, derivative, or prodrug thereof, is administered to the individual at an amount of at most 100 mcg. In some instances, the modulator of the AMPA receptor and/or the 5-HT_{2A} receptor, or the pharmaceutically acceptable salt, solvate, metabolite, derivative, or prodrug thereof, is administered to the individual at an amount of at least 20 micrograms mcg. In some instances, the modulator of the AMPA receptor and/or the 5-HT_{2A} receptor, or the pharmaceutically acceptable salt, solvate, metabolite, derivative, or prodrug thereof, is administered to the individual at an amount of 20-35 mcg, such as, for example, 25-30 mcg. In some embodiments, the modulator of the AMPA receptor and/or the 5-HT_{2A} receptor, or the pharmaceutically acceptable salt, solvate, metabolite, derivative, or prodrug thereof, is administered to the individual at an amount of less than or equal to 60 micrograms (mcg). In some embodiments, the modulator of the AMPA receptor and/or the 5-HT_{2A} receptor, or the pharmaceutically acceptable salt, solvate, metabolite, derivative, or prodrug thereof, is administered to the individual at an amount of 25-60 mcg, such as, for example, 30-60 mcg, or 25-35 mcg.

In some instances, the modulator of the AMPA receptor and/or the 5-HT_{2A} receptor, or the pharmaceutically acceptable salt, solvate, metabolite, derivative, or prodrug thereof, is administered to the individual at an amount of less than or equal to 20 mcg/kg. In some instances, the modulator of the AMPA receptor and/or the 5-HT_{2A} receptor, or the pharmaceutically acceptable salt, solvate, metabolite, derivative, or prodrug thereof, is administered to the individual at an amount from 20-35 mcg/kg, such as from 25-30 mcg/kg. In some embodiments, the modulator of the AMPA receptor and/or the 5-HT_{2A} receptor, or the pharmaceutically acceptable salt, solvate, metabolite, derivative, or prodrug thereof, is administered to the individual at an amount of less than or equal to 60 micrograms (mcg)/kg. In some embodiments, the modulator of the AMPA receptor and/or the 5-HT_{2A} receptor, or the pharmaceutically acceptable salt, solvate, metabolite, derivative, or prodrug thereof, is administered to the individual at an amount from 25-60 mcg/kg, such as from 30-60 mcg/kg, or 25-35 mcg/kg.

In some embodiments, a total daily dose of the modulator of the AMPA receptor and/or the 5-HT_{2A} receptor, or the pharmaceutically acceptable salt, solvate, metabolite, derivative, or prodrug thereof, is administered to the individual at a dose within the window of operability of the active modulator of the AMPA receptor and/or the 5-HT_{2A} receptor in the individual. In some embodiments, a total daily dose of the modulator of the AMPA receptor and/or the 5-HT_{2A} receptor, or the pharmaceutically acceptable salt, solvate, metabolite, derivative, or prodrug thereof, is administered to the individual at a dose within the window of operability, and more than or equal to the therapeutically threshold of the active modulator of the AMPA receptor and/or the 5-HT_{2A} receptor in the individual. In some embodiments, a total daily dose of the modulator of the AMPA receptor and/or the 5-HT_{2A} receptor, or the pharmaceutically acceptable salt, solvate, metabolite, derivative, or prodrug thereof, is administered to the individual at a dose of less than or equal to 30 micrograms (mcg)/day. In some embodiments, a total daily dose of the modulator of the AMPA receptor and/or the 5-HT_{2A} receptor, or the pharmaceutically acceptable salt, solvate, metabolite, derivative, or prodrug thereof, is administered to the individual at a dose from 1-30 mcg/day, such as from 10-30 mcg/day or 20-26 mcg/day). In some embodiments, a total daily dose of the modulator of the AMPA receptor and/or the 5-HT_{2A} receptor, or the pharmaceutically acceptable salt, solvate, metabolite, derivative, or prodrug thereof, is administered to the individual at a dose of less than or equal to 60 micrograms (mcg)/day. In some embodiments, a total daily dose of the modulator of the AMPA receptor and/or the 5-HT_{2A} receptor, or the pharmaceutically acceptable salt, solvate, metabolite, derivative, or prodrug thereof, is administered to the individual at a dose from 25-60 mcg/day, such as from 30-60 mcg/day, or 25-35 mcg/day.

In some embodiments, the method comprises administering the (e.g., active form of the) modulator of the AMPA receptor and/or the 5-HT_{2A} receptor to the individual for a period of time sufficient to increase social behavior in the individual. In some embodiments, the method comprises administering the (e.g., active form of the) modulator of the AMPA receptor and/or the 5-HT_{2A} receptor to the individual for a period of time sufficient to maintain (e.g., increased) social behavior in the individual. In some embodiments, the method comprises administering the (e.g., active form of the) modulator of the AMPA receptor and/or the 5-HT_{2A} receptor to the individual at least every two hours or more, such as, for example, every 12 hours or more, every day or more (e.g., for at least 7 days or more, for at least 14 days or more). In some embodiments, the method comprises administering the (e.g., active form of the) modulator of the AMPA receptor and/or the 5-HT_{2A} receptor to the individual for a period of more than or equal to 1 day.

In some instances, the method comprises administering to the individual the effective amount (e.g., therapeutically effective amount) of the modulator of the AMPA receptor and/or the 5-HT_{2A} receptor, or a pharmaceutically acceptable salt, solvate, metabolite, derivative, or prodrug thereof, at dose such that the concentration of the (e.g., active form of the) modulator of the AMPA receptor and/or the 5-HT_{2A} receptor remains below an unwanted (hallucinogenic) threshold (e.g., within the window of operability) of the (e.g., active form of the) modulator of the AMPA receptor and/or the 5-HT_{2A} receptor in the individual.

In some embodiments, the (e.g., active form of the) modulator of the AMPA receptor and/or the 5-HT_{2A} receptor is administered to the individual at a dose above the therapeutically effective threshold and below an unwanted (hallucinogenic) threshold (e.g., within the window of operability) of the (e.g., active form of the) modulator of the AMPA receptor and/or the 5-HT_{2A} receptor. In some embodiments, the (e.g., active form of the) modulator of the AMPA receptor and/or the 5-HT_{2A} receptor in the individual is administered at a dose above the therapeutically effective threshold of the (e.g., active form of the) modulator of the AMPA receptor and/or the 5-HT_{2A} receptor. In some embodiments, the (e.g., active form of the) modulator of the AMPA receptor and/or the 5-HT_{2A} receptor in the individual is administered at a dose below an unwanted (hallucinogenic) threshold (e.g., within the window of operability) of the (e.g., active form of the) modulator of the AMPA receptor and/or the 5-HT_{2A} receptor. In some embodiments, the (e.g., active form of the) modulator of the AMPA receptor and/or the 5-HT_{2A} receptor in the individual is administered at a dose above the therapeutically effective threshold and a within the window of operability of the (e.g., active form of the) modulator of the AMPA receptor and/or the 5-HT_{2A} receptor.

In some embodiments, the extended period of time is for an entire treatment plan tailored for the individual in need thereof. In some embodiments, the extended period of time is one day, one week, two weeks, one month, six months, one year, or more. In some embodiments, the individual in need thereof is administered the modulator of the AMPA receptor and/or the 5-HT_{2A} receptor, or the pharmaceutically acceptable salt, solvate, metabolite, derivative, or prodrug thereof, for at least one week. In some embodiments, the individual in need thereof is administered the modulator of the AMPA receptor and/or the 5-HT_{2A} receptor, or the pharmaceutically acceptable salt, solvate, metabolite, derivative, or prodrug thereof, daily for a week, every other day for a week, two times a week, once a week, bi-weekly for a month, or the like.

In some embodiments, the (e.g., therapeutically) effective amount of the modulator of the AMPA receptor and/or the 5-HT_{2A} receptor, or a pharmaceutically acceptable salt, solvate, metabolite, derivative, or prodrug thereof, is administered to the individual in need thereof as a controlled release formulation. In some embodiments, the (e.g., therapeutically effective amount of) the modulator of the AMPA receptor and/or the 5-HT_{2A} receptor, or a pharmaceutically acceptable salt, solvate, metabolite, derivative, or prodrug thereof, is administered to the individual in need thereof as an extended release formulation. In some embodiments, the extended release formulation releases the (e.g., active form of the) modulator of the AMPA receptor and/or the 5-HT_{2A} receptor in the individual. In some embodiments, the extended release formulation releases the (e.g., active form of the) modulator of the AMPA receptor and/or the 5-HT_{2A} receptor in the individual such that the concentration of the (e.g., active form of the) modulator of the AMPA receptor and/or the 5-HT_{2A} receptor reaches a Cₘₐₓ below the unwanted (hallucinogenic) threshold (e.g., below a sub-psychotic hallucinogenic threshold) in the individual. In some embodiments, the extended release formulation releases the (e.g., active form of the) modulator of the AMPA receptor and/or the 5-HT_{2A} receptor in the individual such that the (e.g., active form of the) modulator of the AMPA receptor and/or the 5-HT_{2A} receptor reaches a Cₘᵢₙ of at least the therapeutically effective threshold in the individual. In some embodiments, the extended release formulation releases the (e.g., active form of the) modulator of the AMPA receptor and/or the 5-HT_{2A} receptor in the individual such that the concentration of the (e.g., active form of the) modulator of the AMPA receptor and/or the 5-HT_{2A} receptor reaches a Cₘₐₓ within the window of operability and a Cₘᵢₙ of at least the therapeutically effective threshold in the individual.

In some embodiments, the (e.g., therapeutically) effective amount of the modulator of the AMPA receptor and/or the 5-HT_{2A} receptor, or a pharmaceutically acceptable salt, solvate, metabolite, derivative, or prodrug thereof, is administered to the individual in need thereof as an immediate release formulation. In some embodiments, the immediate release formulation releases the (e.g., active form of the) modulator of the AMPA receptor and/or the 5-HT_{2A} receptor in the individual. In some embodiments, the immediate release formulation releases the (e.g., active form of the) modulator of the AMPA receptor and/or the 5-HT_{2A} receptor in the individual such that the concentration of the (e.g., active form of the) modulator of the AMPA receptor and/or the 5-HT_{2A} receptor reaches a Cₘₐₓ below the unwanted (hallucinogenic) threshold (e.g., below a sub-psychotic hallucinogenic threshold) in the individual. In some embodiments, the immediate release formulation releases the (e.g., active form of the) modulator of the AMPA receptor and/or the 5-HT_{2A} receptor in the individual such that the (e.g., active form of the) modulator of the AMPA receptor and/or the 5-HT_{2A} receptor reaches a Cₘᵢₙ, of at least the therapeutically effective threshold in the individual. In some embodiments, the immediate release formulation releases the (e.g., active form of the) modulator of the AMPA receptor and/or the 5-HT_{2A} receptor in the individual such that the concentration of the (e.g., active form of the) modulator of the AMPA receptor and/or the 5-HT_{2A} receptor reaches a Cₘₐₓ within the window of operability and a Cₘᵢₙ of at least the therapeutically effective threshold in the individual.

In some instances, the (e.g., immediate or controlled release) formulation is any formulation provided herein. In some instances, the (e.g., immediate or controlled release) formulation is an oral formulation, an intravenous (IV) formulation, or an intraparietal (IP) formulation. In some embodiments, the oral formulation is in a solid form or a liquid form.

In some embodiments, the controlled release formulation releases the (e.g., active form of the) modulator of the AMPA receptor and/or the 5-HT_{2A} receptor in the individual in need thereof for a period of at least two hours. In some embodiments, the controlled release formulation releases the (e.g., active form of the) modulator of the AMPA receptor and/or the 5-HT_{2A} receptor in the individual in need thereof for a period of at most two hours. In some embodiments, the controlled release formulation releases the (e.g., active form of the) modulator of the AMPA receptor and/or the 5-HT_{2A} receptor in the individual in need thereof for a period of at least one day. In some embodiments, the controlled release formulation releases the (e.g., active form of the) modulator of the AMPA receptor and/or the 5-HT_{2A} receptor in the individual in need thereof for a period of at most one day. In some embodiments, the controlled release formulation releases the (e.g., active form of the) modulator of the AMPA receptor and/or the 5-HT_{2A} receptor in the individual in need thereof for a period of two hours to one week.

In some embodiments, the extended release formulation releases the (e.g., active form of the) modulator of the AMPA receptor and/or the 5-HT_{2A} receptor in the individual in need thereof for a period of at least two hours. In some embodiments, the extended release formulation releases the (e.g., active form of the) modulator of the AMPA receptor and/or the 5-HT_{2A} receptor in the individual in need thereof for a period of at most two hours. In some embodiments, the extended release formulation releases the (e.g., active form of the) modulator of the AMPA receptor and/or the 5-HT_{2A} receptor in the individual in need thereof for a period of at least one day. In some embodiments, the extended release formulation releases the (e.g., active form of the) modulator of the AMPA receptor and/or the 5-HT_{2A} receptor in the individual in need thereof for a period of at most one day. In some embodiments, the extended release formulation releases the (e.g., active form of the) modulator of the AMPA receptor and/or the 5-HT_{2A} receptor in the individual in need thereof for a period of two hours to one week.

In some embodiments, the immediate release formulation releases the (e.g., active form of the) modulator of the AMPA receptor and/or the 5-HT_{2A} receptor in the individual in need thereof for a period of at least two hours. In some embodiments, the immediate release formulation releases the (e.g., active form of the) modulator of the AMPA receptor and/or the 5-HT_{2A} receptor in the individual in need thereof for a period of at most two hours. In some embodiments, the immediate release formulation releases the (e.g., active form of the) modulator of the AMPA receptor and/or the 5-HT_{2A} receptor in the individual in need thereof for a period of at most one day. In some embodiments, the extended release formulation releases the (e.g., active form of the) modulator of the AMPA receptor and/or the 5-HT_{2A} receptor in the individual in need thereof for a period of two hours to one day.

In more specific embodiments, the method, (e.g., pharmaceutical) composition, formulation, or dosage form comprises a pharmaceutically acceptable excipient. In some embodiments, the method, (e.g., pharmaceutical) composition, formulation, or dosage form comprises one or more agents selected from the group consisting of surfactants, preservatives, flavoring agents, sweetening agents, and antifoaming agents.

In certain embodiments, the (e.g., pharmaceutical) composition, formulation, or dosage form is a pharmaceutical composition. In specific embodiments the pharmaceutical composition is a dosage form, such as a discrete dosage form. In more specific embodiments, the pharmaceutical composition is a discrete oral dosage form.

In some embodiments, the therapeutically effective amount of 5-HT receptor agonist is an amount insufficient to elicit an adverse event, such as a hallucinogenic (e.g., an unwanted hallucinogenic) experience (e.g., a psychotic-like hallucinogenic experience) (or other adverse effect) (e.g., in an average adult).

In various embodiments, the (e.g., pharmaceutical) composition is formulated in any suitable manner. For example, in some embodiments, the pharmaceutical composition is a controlled release formulation. In specific embodiments, following administration to an individual in need thereof, the pharmaceutical composition provides a minimum plasma concentration (Cₘₐₓ) of the (e.g., active form of the) modulator of the AMPA receptor and/or the 5-HT_{2A} receptor (e.g., or active metabolite(s) thereof) of at least the therapeutically effective threshold of the (e.g., active form of the) modulator of the AMPA receptor and/or the 5-HT_{2A} receptor in the individual, wherein the minimum plasma concentration (Cₘᵢₙ) is determined at a time between 2 hours and 12 hours (or between 2 hours and 24 hours, or between 2 hours and 48 hours, or between 2 hours and 72 hours, or the like) after administration to the individual.

In specific embodiments, the formulations provided herein provide controlled release such that the minimum plasma concentration (Cₘᵢₙ) is determined at a time between 24 and 48 hours after administration. For example, if plasma concentration of the agent(s) continues to decline over time, then after 48 hours after administration, plasma levels of the agent(s) are at least the value indicated after 48 hours.

In certain preferred embodiments, the pharmaceutical composition or dosage form is formulated for oral administration.

In various embodiments herein, a composition (e.g., pharmaceutical composition, dosage form, combination or formulation) provided herein is administered at any frequency. For example, in some embodiments, a single dose or repeated doses are provided. In other embodiments, the composition is or is formulated to be administered to a subject in need thereof about once a week. In other embodiments, the composition is or is formulated to be administered to a subject in need thereof about once every two weeks. In various other embodiments, the composition is or is formulated for twice daily, once daily, twice weekly, thrice weekly, or the like administration.

In various embodiments, such a composition is formulated to have any of the components and/or features as described for a composition provided herein, such as described above. In some embodiments, such a composition comprises one or more pharmaceutically acceptable excipient (e.g., a filler, binder, suspending agent, disintegrant, lubricant, surfactant, preservative, flavoring agent, sweetener, or a combination of two or more thereof).

In certain embodiments, any composition provided herein is formulated and/or packaged to be repeatedly administered to a subject in need thereof about once a week (or more frequently, such as two or three times a week, daily, twice daily, or the like). In some embodiments, any composition provided herein is formulated and/or packaged to be repeatedly administered to a subject in need thereof about once every two weeks (or less frequently). In certain embodiments, any composition provided herein is formulated and/or packaged to be repeatedly administered to a subject in need thereof about once every month.

Provided in certain embodiments herein is a method comprising administering to the subject any composition (e.g., pharmaceutical combination, composition, dosage form, or formulation) provided herein. In specific embodiments, such a method comprises administering a therapeutically effective amount of the (e.g., active form of the) modulator of the AMPA receptor and/or the 5-HT_{2A} receptor as an extended release dosage form, such as providing a composition and/or effect described herein.

In specific embodiments, such a method is provided for of treating or managing a neurological condition or the symptoms thereof in a subject in need thereof (e.g., in a subject suffering from or susceptible to the neurological condition).

In some embodiments, the therapeutically effective amount of the (e.g., active form of the) modulator of the AMPA receptor and/or the 5-HT_{2A} receptor is an amount insufficient to elicit an adverse event, such as a hallucinogenic (e.g., an unwanted hallucinogenic) experience (e.g., a psychotic-like hallucinogenic experience) (or other adverse effect) (e.g., in an average adult).

In certain preferred embodiments, the method comprises oral administration.

In various embodiments, methods provided herein are suitable for treating any suitable disease, disorder, or condition, such as a neurological condition, such as a neurological disorder, or symptoms thereof. In specific embodiments, the condition is social dysfunction or social behavior. In specific embodiments, the neurological condition is a neurodegenerative condition or a neurodevelopmental condition. In specific embodiments, the neurological condition is a neurocognitive disorder. In some embodiments, symptoms of the neurological condition are physical, behavioral, emotional, mental or a combination thereof. In certain embodiments, the neurological condition is an addictive disorder. In some embodiments, the neurological condition is an eating disorder or an auditory disorder. In some embodiments, the neurological condition is depression, bipolar disorder, post-traumatic stress disorder (PTSD), panic disorder, phobia, schizophrenia, psychopathy, or antisocial personality disorder. In certain embodiments, the neurological condition is an impulsive disorder. In some embodiments, the impulsive disorder is attention deficit hyperactivity disorder (ADHD), Tourette's syndrome or autism. In some embodiments, the neurological condition is a personality disorder (e.g., conduct disorder, antisocial personality, or aggressive behavior). In some embodiments, the disease, disorder, or condition is Alzheimer's disease or Parkinson's disease.

In various embodiments, the combination of agents is administered in any suitable formulation or form, such as in combination with one or more agents selected from the group consisting of surfactants, preservatives, flavoring agents, sweetening agents, and antifoaming agents. In certain embodiments, administration is via use of an oral formulation.

In certain embodiments, administration to a subject in need thereof occurs no more frequently than once a day (e.g., no more frequently than once every other day, no more frequently than once every third day, no more frequently than twice a week, no more frequently than once a week, no more frequently than once every two weeks, or the like). In some embodiments, administration to a subject in need thereof occurs once a day, every alternate day, three times a week, twice a week, once a week, every other week, two weeks per month, three weeks per month, once a month, twice a month or three times per month. In specific embodiments, administration is about once a day. In other specific embodiments, administration is about every alternate day. In still other specific embodiments, administration is about once a week. In yet other specific embodiments, administration is about once every two weeks or more.

In various embodiments, administration continues for any suitable length of time, such as at least 1 month, 2 months, 3 months, 4 months, 5 months, 6 months, 7 months, 8 months, 9 months, 10 months, 11 months, 12 months, 18 months, 2 years, or 3 years.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows administration of repeated doses of a modulator of the AMPA receptor and/or the 5-HT_{2A} receptor (e.g., LSD), or a pharmaceutically acceptable salt, solvate, metabolite, derivative, or prodrug thereof exerts prosocial effects in the direct social interaction (DSI) and in the three chambers test (TCT). FIG. 1A shows that repeated (e.g., a 7 day-treatment regimen) administration of a modulator of the AMPA receptor and/or the 5-HT_{2A} receptor (e.g., LSD), or a pharmaceutically acceptable salt, solvate, metabolite, derivative, or prodrug thereof but not acute administration increases interaction time (s) toward the unfamiliar mouse compared to the vehicle (veh). FIG. 1B shows repeated administration (e.g., a 7 day-treatment regimen) of a modulator of the AMPA receptor and/or the 5-HT_{2A} receptor (e.g., LSD), or a pharmaceutically acceptable salt, solvate, metabolite, derivative, or prodrug thereof promotes preference for social novelty since increase the sniffing time toward the novel unfamiliar mouse compared to the vehicle (veh). * P<0.05 and **P<0.01, ***P<0.001. N.S.=not significant.

FIG. 2 shows the effects of repeated LSD treatment in mice in both a light-dark box test and a novelty suppressed feeding test following chronic restraint stress. FIG. 2A shows that LSD normalized the transition between the dark and the light compartment in the light-dark box test after chronic restraint stress. FIG. 2B shows that LSD decreased latency to feed in the novelty suppressed feeding test after chronic restraint stress.

FIG. 3 shows the increase in head twitches as an acute (e.g., single) dose of LSD is increased.

### DETAILED DESCRIPTION

### Social Behavior Disorders

Autism spectrum disorder (ASD) is a neurodevelopmental disorder characterized by severe social interaction and cognitive deficits (e.g., characterized by persistent deficits in social interaction, repetitive patterns of behavior, and interests or activities, cognitive deficits). The worldwide prevalence of ASD ranges from between 1-2%, with rates in Canada and the U.S. being at 1 in 66 and 1 in 59, respectively. ASD incidence is 4 times higher in males than females. Individuals suffering from ASD tend to have increased risk of psychiatric problems such as, for example, anxiety, depression, obsessive-compulsive, and eating disorders. Potentiation of both glutamatergic and serotonergic transmission are implicated in ASD. In addition, mTOR signaling cascade has been demonstrated to exert a pivotal role in ASD.

The term “social behavior,” which is used interchangeably with “SB,” as used herein, generally refers to interactions among individuals, normally within the same species, that can be beneficial to one individual or a group of individual. In certain instances, SB in mammals is mediated by the prefrontal cortex (PFC) (e.g., since PFC pathology may generate apathy and impaired SB). As a non-limiting example, disrupted SB is prevalent in various mental illnesses, including, but not limited to, Autism Spectrum Disorder (ASD) and Social Anxiety Disorders (SAD).

Severe dysfunctions of SB can lead to psychiatric diseases such as, for example, ASD (e.g., characterized by deficient reciprocity and communication and unusual, restrictive, and repetitive behavior, affecting 1.5% of the population) and social anxiety (formerly termed social phobia) (e.g., the fear of being judged and evaluated negatively by other people, leading to feelings of inadequacy, inferiority, self-consciousness, and embarrassment, affecting about 7% of the population in Canada). There are currently no treatments for autism or social phobias, and doctors use off-label drugs such as, for example, antidepressants or atypical antipsychotics to treat these conditions.

### Treatment Regimen

Lysergic acid diethylamide (LSD) is a psychedelic compound which has been demonstrated to safely relieve anxiety disorders in patients with life-threatening illnesses (when used in a controlled setting). In addition, LSD has been demonstrated to increase social behavior in healthy volunteers. For example, in healthy volunteers, LSD produces feelings of happiness, trust, closeness to others, enhances explicit and implicit emotional empathy, and attenuates emotions during the recognition of fearful faces. Positive mood changes and altruistic/positive social effects were reported after 200 μg of LSD; increased optimism, mood and trait openness were observed 2 weeks after LSD administration (e.g., suggesting that LSD can induce long-lasting neuroplasticity).

LSD acts as a partial agonist of the 5-hydroxytryptamine 2A (5-HT_{2A}) receptor with a binding affinity of 2.5 nM (ki). LSD also displays affinity as a partial agonist for the 5-hydroxytryptamine 1A (5-HT_{1A}) receptor (ki:1.1 nM) and the dopamine D2 receptor (ki: 0.025 μM). In some instances, (e.g., hallucinogenic) doses (e.g., 5-30 μg/kg) of LSD acts on the 5-HT system by decreasing the activity of 5-HT neurons originating in the Dorsal Raphe Nucleus (DRN) (e.g., in mice). In some instances, (e.g., hallucinogenic) doses (e.g., 30-120 μg/kg, 60-120 μg/kg) inhibit the dopaminergic system, thus leading to psychotic-like hallucinogenic symptoms, such as, for example, hyper-locomotion, stereotypical behavior, head-twitches and impaired pre-pulse inhibition.

In some embodiments, the method provided herein comprises administering to an individual a (e.g., therapeutically) effective amount of one or more modulator of an AMPA receptor and a 5-HT (e.g., a 5-HT_{2A}) receptor (e.g., lysergic acid (LSD)), or a pharmaceutically acceptable salt, solvate, metabolite, derivative, or prodrug thereof (e.g., for an extended period of time (e.g., for repeating days)).

In some instances, the modulator of the AMPA receptor and/or the 5-HT_{2A} receptor, or the pharmaceutically acceptable salt, solvate, metabolite, derivative, or prodrug thereof, is administered to the individual at an amount of at most 200 micrograms (mcg). In some instances, the modulator of the AMPA receptor and/or the 5-HT_{2A} receptor, or the pharmaceutically acceptable salt, solvate, metabolite, derivative, or prodrug thereof, is administered to the individual at an amount of at most 100 mcg.

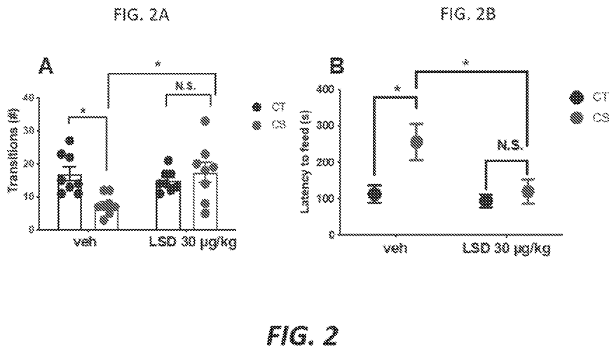

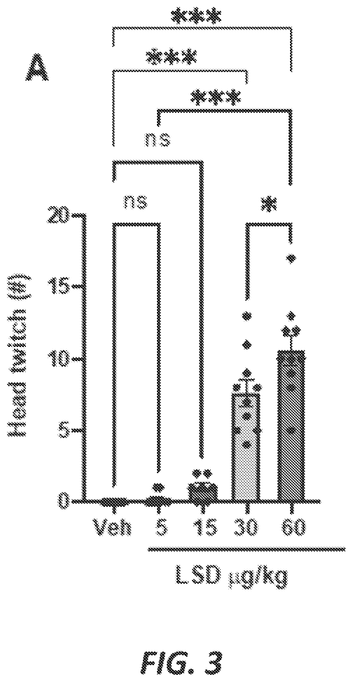

In some instances, acute (e.g., daily) doses of about 30 mcg/kg or more provide adverse events (e.g., hallucinations, such as, for example, head-twitches) in an individual (FIG. 3). In some instances, acute (e.g., daily) doses of about 30 mcg/kg or more, such as, 60 mcg/kg, provide adverse events (e.g., hallucinations, such as, for example, head-twitches) in an (FIG. 3). In some instances, acute (e.g., daily) doses of about 15 mcg/kg or less do not provide adverse events (e.g., hallucinations, such as head-twitches) in an individual (FIG. 3). In some instances, acute (e.g., daily) doses of about 15 mcg/kg or less do not provide adverse events (e.g., hallucinations, such as head-twitches) in an individual and are too low to provide a therapeutic effect (FIG. 3). In some instances, acute (e.g., daily) doses of about 30 mcg/kg provide adverse events (e.g., hallucinations, such as head-twitches) in an individual (FIG. 3). In some instances, acute (e.g., daily) doses of about 30 mcg/kg provide adverse events (e.g., hallucinations, such as head-twitches) in an individual and provide a therapeutic effect (FIG. 3 and FIG. 2A).

In some embodiments, provided herein is a method of treating and/or maintaining social behavior disorders, comprising administering repeated doses of LSD to an individual in need thereof.

In some instances, the individual is a mammal, such as a human, a rodent (e.g., a mouse or rat), a primate (e.g., a monkey), a dog, or the like. In some instances, the individual is a rodent. In some instances, the individual is a mouse. In some instances, the individual is a human. In some instances, the individual is a primate.

In some embodiments, provided herein is a method of improving social interaction in mice by administering a repeated (e.g., 30 mcg/kg) dose of LSD (e.g., at least 1 μg/kg (e.g., 20 g/kg or more, 25 μg/kg or more, 30 μg/kg or more, 35 μg/kg or more, 40 μg/kg or more, 45 μg/kg or more, 50 μg/kg or more, 55 μg/kg or more, or 60 μg/kg or more) per day (e.g., for 7 days)) to an individual in need thereof. In some embodiments, provided herein is a method of treating ASD or autism-like phenotypes (e.g., social interaction, repetitive patterns of behavior, interests or activities, cognitive deficits, eating disorders, anxiety, depression, and obsessive-compulsive) by administering repeating doses of LSD to an individual in need thereof.

### Single Administration

In some embodiments, a modulator of the 5-HT_{2A} receptor and/or the AMPA receptor is administered to an individual in need thereof. In some instances, the method comprises administering at least one dose of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof). In some embodiments, a modulator of the 5-HT_{2A} receptor and/or the AMPA receptor (e.g., LSD) is administered to an individual in need thereof once (e.g., at a dose of at least 1 μg/kg (e.g., 20 μg/kg or more, 25 μg/kg or more, 30 μg/kg or more, 35 μg/kg or more, 40 μg/kg or more, 45 μg/kg or more, 50 μg/kg or more, 55 μg/kg or more, or 60 μg/kg or more)). In some embodiments, a modulator of the 5-HT_{2A} receptor and/or the AMPA receptor (e.g., LSD) is administered to an individual in need thereof at a dose (e.g., of at least 1 μg/kg (e.g., 20 μg/kg or more, 25 μg/kg or more, 30 μg/kg or more, 35 μg/kg or more, 40 μg/kg or more, 45 μg/kg or more, 50 μg/kg or more, 55 μg/kg or more, or 60 μg/kg or more)) sufficient to decrease the 5-HT firing activity of the Dorsal Raphe Nucleus (DRN). In some embodiments, the modulator of the 5-HT_{2A} receptor and/or the AMPA receptor is LSD.

In some embodiments, provided herein is a method for treating or managing a mental, a behavioral, or a neuropsychiatric condition, or the symptoms thereof, in an individual in need thereof (e.g., in an individual suffering from or susceptible to the mental, the behavioral, or the neuropsychiatric condition) by administering to the individual a single dose of a modulator of the 5-HT_{2A} receptor and/or the AMPA receptor (e.g., LSD)) to an individual in need thereof.

In some embodiments, provided herein is a method for increasing social interaction or decreasing social dysfunction in an individual (e.g., in an individual suffering from or susceptible to anxiety (e.g., social anxiety, anxiety disorders, generalized anxiety), fear, phobia (e.g., a social phobia), constructive impulsivity, autism spectrum disorder (ASD), or depression) by administering a single dose of a modulator of the 5-HT_{2A} receptor and/or the AMPA receptor (e.g., LSD)) to the individual in need thereof.

In some embodiments, provided herein is a method for treating or managing a neurodegenerative condition or a neurodevelopmental condition (e.g., that affects cognition), or the symptoms thereof, in an individual in need thereof (e.g., in an individual suffering from or susceptible to the neurodegenerative condition or the neurodevelopmental condition) by administering a single dose of a modulator of the 5-HT_{2A} receptor and/or the AMPA receptor (e.g., LSD)) to the individual in need thereof.

In some embodiments, provided herein is a method for improving despair, anhedonia, anxiety, stereotypic behavior, and social behavior in an individual in need thereof (e.g., by administering to the individual a single dose of a modulator of the 5-HT_{2A} receptor and/or the AMPA receptor (e.g., LSD)) in the individual in need thereof. In some embodiments, provided herein is a method for improving social behavior in an individual in need thereof (e.g., by administering a single dose of a modulator of the 5-HT_{2A} receptor and/or the AMPA receptor (e.g., LSD)) to the individual in need thereof.

In some embodiments, the mental, the behavioral, or the neuropsychiatric condition is a social behavior (e.g., a social anxiety disorder). In some embodiments, the mental, the behavioral, or the neuropsychiatric condition is an anxiety disorder or a depression disorder. In some embodiments, the mental, the behavioral, or the neuropsychiatric condition is depression. In some embodiments, the mental, the behavioral, or the neuropsychiatric condition is anxiety. In some embodiments, the mental, the behavioral, or the neuropsychiatric condition is social anxiety.

### Repeated Administration

In some embodiments, a modulator of the 5-HT_{2A} receptor and/or the AMPA receptor (e.g., LSD) is administered (e.g., i.p.) to a subject (e.g., an individual or a mouse) provided herein at least once per day (e.g., at a dose of at least 1 μg/kg/day (e.g., 20 μg/kg/day or more, 25 μg/kg/day or more, 30 μg/kg/day or more, 35 μg/kg or more, 40 μg/kg or more, 45 μg/kg or more, 50 μg/kg or more, 55 μg/kg or more, or 60 μg/kg or more)) (e.g., for at least two days, such as, for example, for 2 days, for 3 days, for 4 days, 5 days, 6 days, 7 days, or more). In some embodiments, the modulator of the 5-HT_{2A} receptor and/or the AMPA receptor (e.g., LSD) is administered on a first day and a second day. In some embodiments, the second day is at least one day after (e.g., at least two days after, at least three days after, at least four days after, or at least 7 days after) the first day.

In some embodiments, provided herein is a method for treating or managing a mental, a behavioral, or a neuropsychiatric condition, or the symptoms thereof, in an individual in need thereof (e.g., in an individual suffering from or susceptible to the mental, the behavioral, or the neuropsychiatric condition) by administering to the individual repeated doses (e.g., at least two doses (e.g., 2 doses, 3 doses, 4 doses, 5 doses, 6 doses, 7 doses, or more) for at least two days, such as, for example, for 2 days, for 3 days, for 4 days, 5 days, 6 days, 7 days, or more) of a modulator of the 5-HT_{2A} receptor and/or the AMPA receptor (e.g., LSD)) in an individual in need thereof.

In some instances, the method comprises acute administration of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof). In some instances, the method comprises sub-chronic administration of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof). In some instances, the method comprises chronic administration of the effective amount of the modulator of the AMPA receptor and/or the 5-HT_{2A} receptor (e.g., LSD), or a pharmaceutically acceptable salt, solvate, metabolite, derivative, or prodrug thereof, to the individual (e.g., in need thereof).

In some embodiments, provided herein is a method for increasing social interaction or decreasing social dysfunction in an individual (e.g., in an individual suffering from or susceptible to anxiety (e.g., social anxiety, anxiety disorders, generalized anxiety), fear, phobia (e.g., a social phobia), constructive impulsivity, autism spectrum disorder (ASD), or depression) by administering to the individual repeated doses (e.g., at least two doses (e.g., 2 doses, 3 doses, 4 doses, 5 doses, 6 doses, 7 doses, or more) for at least two days, such as, for example, for 2 days, for 3 days, for 4 days, 5 days, 6 days, 7 days, or more) of a modulator of the 5-HT_{2A} receptor and/or the AMPA receptor (e.g., LSD)) to the individual in need thereof.

In some embodiments, provided herein is a method for treating or managing a neurodegenerative condition or a neurodevelopmental condition (e.g., that affects cognition), or the symptoms thereof, in an individual in need thereof (e.g., in an individual suffering from or susceptible to the neurodegenerative condition or the neurodevelopmental condition) by administering to the individual repeated doses (e.g., at least two doses (e.g., 2 doses, 3 doses, 4 doses, 5 doses, 6 doses, 7 doses, or more) for at least two days, such as, for example, for 2 days, for 3 days, for 4 days, 5 days, 6 days, 7 days, or more) of a modulator of the 5-HT_{2A} receptor and/or the AMPA receptor (e.g., LSD)) to the individual in need thereof.

In some embodiments, provided herein is a method for improving despair, anhedonia, anxiety, stereotypic behavior, and social behavior in an individual in need thereof by administering to the individual repeated doses (e.g., at least two doses (e.g., 2 doses, 3 doses, 4 doses, 5 doses, 6 doses, 7 doses, or more) for at least two days, such as, for example, for 2 days, for 3 days, for 4 days, 5 days, 6 days, 7 days, or more) of a modulator of the 5-HT_{2A} receptor and/or the AMPA receptor (e.g., LSD)) to the individual in need thereof. In some embodiments, provided herein is a method for improving social behavior in an individual in need thereof (e.g., by administering to the individual repeated doses of a modulator of the 5-HT_{2A} receptor and/or the AMPA receptor (e.g., LSD)) to the individual in need thereof.

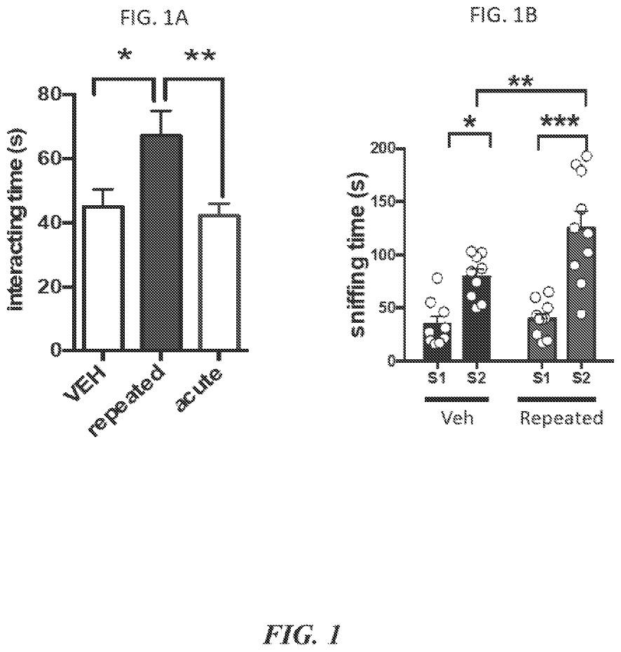

In some embodiments, administering repeated doses of the 5-HT_{2A} receptor and/or the AMPA receptor (e.g., LSD)) increases social interaction (e.g., with a stranger), reduces anxiety and/or depression in an individual in need thereof more than administering a single dose of the 5-HT_{2A} receptor and/or the AMPA receptor (e.g., LSD)) in the individual (e.g., FIG. 1 (e.g., FIG. 1A and FIG. 1B) and FIG. 2 (FIG. 2A and FIG. 2B)). In some embodiments, administering repeated doses of the 5-HT_{2A} receptor and/or the AMPA receptor (e.g., LSD)) to an individual in need thereof increases social interaction (e.g., with a stranger), reduces anxiety and/or depression in the individual.

### EXAMPLES

These examples are provided for illustrative purposes only and not to limit the scope of the claims provided herein.

### Example 1: Repeated LSD Treatment Increases Social Interaction in the Direct Social Interaction (DSI) and in the Three Chambers Test (TCT)

Repeated administration of LSD (e.g., 25-35 μg/kg, per day, for 7 days) increased social interaction in male C57BL/6J mice compared with veh (e.g., saline). The increase in social interaction was observed 24 hours after the last injection in two validated paradigms of social behavior: the direct social interaction test (FIG. 1A) and the three-chamber test (FIG. 1B). Lower doses (e.g., 5, 10 μg/kg for 3 days) produced a non-significant increase in social interaction in mice. Interestingly, in a separate study, lower acute (e.g., single) doses (e.g., 15 μg/kg or less) did not provide hallucinogenic effects (e.g., head-twitches) in mice (FIG. 3). Similarly, a single administration of LSD did not produce any effect on social behavior (FIG. 1A) of LSD in mice. Similarly, repeated administration of LSD (e.g., 25-35 μg/kg, per day, for 7 days) promoted the preference for the social novelty by increasing the sniffing time toward a novel mouse in the three chambers apparatus (FIG. 1B). In addition, these results were coupled with electrophysiological experiments showing that this (e.g., repeated) regimen of LSD potentiates both glutamatergic and serotonergic neurotransmission in the medial prefrontal cortex (mPFC) and in the DRN (e.g., two brain regions involved in Autism Syndrome Disorder (ASD). In addition, LSD increased the phosphorylation levels of the mammalian target of rapamycin complex (mTOR) in the mPFC.

### Example 2: Repeated LSD Treatment Prevents Chronic Stress-Induced Anxiety-Like Behavior in the Light-Dark Box Test and in the Novelty Suppressed Feeding Test

### Chronic Restraint Stress

A paradigm of repeated stress-induced anxiety-like behavior was employed, in order to mimic anxious states induced by chronic stressor exposure in humans. Chronic restraint stress was chosen for its ability to reliably and relatively quickly induce anxiety-like behavior. Briefly, mice were placed in well ventilated plexiglass restrainers for 2 hours per day, for 15 days, then returned to a horizontal resting position on an open bench. During the procedure, the animals had no access to food and water. Control mice were left undisturbed in their home cage. This regimen duration was chosen given that, in pilot experiments, it represented the minimum duration of restraint stress which exacerbated anxiety-like behavior.

### Novelty Suppressed Feeding Test

This procedure was used to measure neohypophagia (the anxiety-induced inhibition of feeding upon exposure to a novel environment). This test is used to validate the acute effects of putative anxiolytics. Mice were food-deprived for 48-hours; subsequently, each mouse was placed in a brightly illuminated (100 W, 350 Ix) open arena (40×40 cm, white-painted floor with walls 30 cm high), containing standard chow (3 pellets) in the center of the arena. The latency to initiate feeding (in seconds) was used as an index of anxiety-like behavior. The cut-off time was 600 seconds, after which mice that did not feed were excluded from the analysis. To ensure that the mice were indeed hungry, feeding latency was also observed in the home cage containing 3 pellets on the floor of the cage; the session was terminated immediately after the mice initiated feeding.

### Light-Dark Box Test

For the light-dark box test, an apparatus consisting of a light, open topped, opaque, plexiglass box (27×27×30 cm) connected to a dark, closed topped, opaque, plexiglass box (18×27×30 cm) was used. The light and dark boxes were connected by a small opening (12×5 cm) which allowed the mouse to cross between the two compartments. The light box was illuminated by a 100 W desk lamp. Each mouse was placed in the illuminated box and observed for 5 min. When the four paws were in the light box, the mouse was considered to be in the light compartment. Time spent in the light compartment, and number of crossings between the light and the dark compartments were used as indexes of anxiety-like behavior.

### Results

Repeated LSD administration (e.g., 30 μg/kg) prevents chronic stress-induced anxiety-like behavior in the light-dark box test and in the novelty suppressed feeding test (FIG. 2A and FIG. 2B). Notably, in a separate study, an acute (e.g., single) dose of LSD (e.g., 30 μg/kg) produces adverse events (e.g., hallucinations, such as, for example, head twitches) (FIG. 3). Further, an acute (e.g., single) dose of LSD (e.g., 60 μg/kg) produces adverse events (e.g., hallucinations, such as, for example, head twitches) (FIG. 3).

To further assess the protective effects of the effective LSD dose (30 μg/kg) over stress-induced anxiety-like behavior, another cohort of LSD-treated mice and controls were exposed to chronic restraint stress and tested in the light-dark box test and novel suppressed feeding test.

As illustrated in FIG. 2A, mice tested in the light-dark box test produced a decreased number of transitions between the light and dark compartments (FIG. 2A, stress factor, F (1, 28)=2.706, p=0.1111; treatment factor, F (1, 28)=3.568, p=0.0693; interaction stress×treatment, F (1, 28)=8.554, p=0.0068). Post-hoc analysis revealed that stressed mice treated with LSD showed a number of entries similar to the control mice treated with the vehicle (p<0.05). In addition, the same cohort of mice was tested in the novel suppressed feeding test. As shown in FIG. 2B, following exposure to a novel environment, chronic restraint stress mice showed an increased latency to feed in the arena compared to CTL (FIG. 2B, stress factor, F (1, 28)=6.564, p=0.0159; treatment factor, F (1, 28)=5.436, p=0.0269; interaction stress×treatment, F (1, 28)=3.163, p=0.0005), thus indicating that the stress condition exacerbated the anxiety-like behavior triggered by exposure to a novel environment. Post-hoc analysis revealed that chronic restraint stress mice treated with LSD had a reduced latency to feed compared to vehicle-treated stressed mice (p<0.05).

Overall, these data indicate that the repeated treatment with LSD at the dose of 30 μg/kg, prevents the development of anxiety-like behavior due to chronic stress exposure.

While preferred embodiments have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments described herein might be employed in practicing current disclosure.

1-23. (canceled)

24. A method for improving the symptoms of a neuropsychiatric condition, in an individual in need thereof, the method comprising:
a. administering to the individual a therapeutically effective amount of lysergic acid diethylamide (LSD), or a pharmaceutically acceptable salt thereof, on a first day; and
b. administering to the individual the therapeutically effective amount of the lysergic acid diethylamide (LSD), or a pharmaceutically acceptable salt thereof, on a second day, the second day being at least one day after the first day.
wherein at least one of the therapeutically effective amount of lysergic acid diethylamide (LSD) is insufficient to provide a hallucinogenic experience.

25. The method of claim 24, wherein the second day is at least 1 day after the first day.

26. The method of claim 24, wherein the second day is at least 7 days after the first day.

27. The method of claim 24, wherein the method further comprises administering to the individual the therapeutically effective amount of the LSD, or a pharmaceutically acceptable salt thereof on a third day, wherein the third day is between the first and second day.

28. The method of claim 24, wherein the method further comprises administering to the individual the therapeutically effective amount of the LSD, or a pharmaceutically acceptable salt thereof on a third day, wherein the third day is after the second day.

29. The method of claim 24, wherein the first day is no less than one day apart from the second day and the second day is no less than one day apart from the third day.

30. The method of claim 24, wherein the therapeutically effective amount of the LSD is administered at least once daily for at least two days.

31. The method of claim 24, wherein the therapeutically effective amount of the LSD, or the pharmaceutically acceptable salt thereof, is less than or equal to 200 mcg.

32. The method of claim 24, wherein the therapeutically effective amount of the LSD, or the pharmaceutically acceptable salt thereof, is less than or equal to 100 mcg.

33. The method of claim 24, wherein the therapeutically effective amount of the LSD, or the pharmaceutically acceptable salt thereof, is less than or equal to 60 mcg.

34. The method of claim 32, wherein the therapeutically effective amount of the LSD, or the pharmaceutically acceptable salt thereof, is about 20 mcg to about 50 mcg.

35. The method of claim 34, wherein the therapeutically effective amount of the LSD, or the pharmaceutically acceptable salt thereof, is about 25 mcg to about 35 mcg.

36. The method of claim 24, wherein the therapeutically effective amount of the LSD, or a pharmaceutically acceptable salt thereof, is administered to the individual in need thereof as a controlled release formulation to the individual in need thereof.

37. The method of claim 24, wherein the neuropsychiatric condition is an anxiety or depression disorder.

38. The method of claim 37, wherein the anxiety disorder is social anxiety.

39. The method of claim 37, wherein the anxiety disorder is generalized anxiety disorder.

40. The method of claim 24, wherein the symptoms of the neuropsychiatric condition are physical, behavioral, emotional, mental, or a combination thereof.

41. The method of claim 24, wherein the therapeutically effective amount of the LSD, or a pharmaceutically acceptable salt thereof, is administered as an oral formulation, intravenous formulation, or an intraparietal formulation to the individual in need thereof.

42. The method of claim 41, wherein the oral formulation is in a solid form or a liquid form.
